(19) United States
(12) Patent Application Publication
(10) Pub. No.: US 2026/0151527 A1
(43) Pub. Date: Jun. 4, 2026

(54) Aromatherapy device

(71) Applicant: Dongguan Xiaofan Innovation Technology Co., Ltd., Dongguan (CN)

(72) Inventor: Zhiting Li, Loudi (CN)

(21) Appl. No.: 19/015,683

(22) Filed: Jan. 10, 2025

(30) Foreign Application Priority Data
Dec. 4, 2024 (CN) 2024117758848

(51) Int. Cl. A61L 9/03

(52) U.S. Cl. CPC A61L 9/032; A61L 2209/133

(57) ABSTRACT

The present disclosure relates to an aromatherapy device, comprising detachably connected two parts, the first part being an aromatherapy storage module, the aromatherapy storage module comprising a first opening for diffusing aromatherapy vapor; the second part being a host module, the host module comprising a support frame and a fan unit matched with the aromatherapy storage module; wherein, the aromatherapy storage module is installed on the host module to form an air duct unit and an energizing unit, and the air duct unit is configured for the passage of wind generated by the fan unit. The host module comprises a first air duct, and the wind generated by the fan unit enters the aromatherapy storage module through the first air duct.

### CLAIMS OF PRIORITY

This patent application claims priority from:
(1) CN invention patent application number is 2024117758848 with a filing date of Dec. 4, 2024 and DAS code of 7728.

### FIELD

The present disclosure relates to the technical field of aromatherapy, particularly an aromatherapy device.

### BACKGROUND

With the improvement of people's living standards, more and more people have begun to pursue higher quality and more refined lifestyles. In daily life, due to the mental pressure caused by work and external environment, more people choose to use essential oil products to relieve tension and soothe the spirit. An aromatherapy diffuser is an aroma diffuser that uses an atomization device to decompose dissolved plant essential oil into a mist to diffuser in the surrounding air. Aromatherapy diffusers have the effect of purifying the air, improving the environment, calming emotions, relieving stress and are currently widely used. Due to the corrosiveness of essential oils, when the current aromatherapy diffusers are carried or used, the oil may leak and drip on a desktop or in a luggage or pocket, causing inconvenience to the user. There is an urgent need in the art for an aromatherapy device that is compact, easy to carry and replace aromatherapy essential oils.

### SUMMARY

In view of the above and other more ideas, the present disclosure is proposed.

According to one aspect of the present disclosure, an aromatherapy device is provided, the aromatherapy device comprising:
an aromatherapy storage module, the aromatherapy storage module comprising a first opening for diffusing aromatherapy vapor;
a host module, the host module comprising a support frame, a fan unit and a first air duct that are matched with the aromatherapy storage module, wind generated by the fan unit entering the aromatherapy storage module through the first air duct;
wherein, the aromatherapy storage module and the host module are detachable.

In one embodiment, the aromatherapy storage module further comprises a storage bin and an atomization bin.

In one embodiment, the aromatherapy storage module further comprises a first oil-absorbing unit provided at one end of the storage bin, an aromatherapy unit provided in the storage bin, a second oil-absorbing unit provided at the other end of the storage bin and opposite to the first oil-absorbing unit, a heating unit for heating the aromatherapy unit and extending into the storage bin and a first housing, the first oil-absorbing unit, the aromatherapy unit, the second oil-absorbing unit and the heating unit being provided inside the first housing.

In one embodiment, the aromatherapy storage module further comprises a second air duct opposite to the first air duct.

In one embodiment, the heating unit is a hollow round pipe, one end of the heating unit is communicated with the second air duct, one end of the heating unit away from the second air duct extends into the aromatherapy unit, and a hollow inner cavity of the heating unit acts as a third air duct.

In one embodiment, the aromatherapy storage module further comprises a first energizing unit, and the first energizing unit is connected with the heating unit and used for providing electric power to heat the heating unit.

In one embodiment, the host module comprises a second energizing unit corresponding to the first energizing unit, and when the aromatherapy storage module is installed on the host module, the first energizing unit abuts against the second energizing unit.

In one embodiment, the first housing of the aromatherapy storage module is provided with a first guiding unit, and the support frame of the host module is provided with a second guiding unit matched with the first guiding unit.

In one embodiment, the host module further comprises a power supply unit, and the power supply unit is connected with the second energizing unit.

In one embodiment, the host module comprises a second housing and a third housing, the third housing encloses a groove body to act as the support frame, and when the aromatherapy storage module is installed on the host module, the third housing matches with the first housing.

In one embodiment, the main body of the aromatherapy unit is an oil storage cotton with an air hole, the oil storage cotton fills the storage bin, and the wind generated by the fan unit enters the atomization bin through the first air duct, the second air duct, the third air duct and the oil storage cotton.

In one embodiment, the first guiding unit is a fin higher than a surface of the first housing.

In one embodiment, the second guiding unit is a channel matched with the fin.

In one embodiment, the host module further comprises a circuit board unit.

According to another aspect of the present disclosure, an aromatherapy device is provided, the aromatherapy device comprising detachably connected two parts, the first part being an aromatherapy storage module, the aromatherapy storage module comprising a first opening for diffusing aromatherapy vapor;
the second part being a host module, the host module comprising a support frame and a fan unit that are matched with the aromatherapy storage module;
wherein, the aromatherapy storage module is installed on the host module to form an air duct unit and an energizing unit, and the air duct unit is configured for the passage of wind generated by the fan unit.

In one embodiment, the host module comprises a first air duct, and the wind generated by the fan unit enters the aromatherapy storage module through the first air duct.

In one embodiment, the aromatherapy storage module further comprises a storage bin and an atomization bin.

In one embodiment, the aromatherapy storage module further comprises a first oil-absorbing unit provided at one end of the storage bin, an aromatherapy unit provided in the storage bin, a second oil-absorbing unit provided at the other end of the storage bin and opposite to the first oil-absorbing unit, a heating unit for heating the aromatherapy unit and extending into the storage bin and a first housing, the first oil-absorbing unit, the aromatherapy unit, the second oil-absorbing unit and the heating unit being provided inside the first housing.

In one embodiment, the aromatherapy storage module further comprises a second air duct opposite to the first air duct.

In one embodiment, the heating unit is a hollow round pipe, one end of the heating unit is communicated with the second air duct, one end of the heating unit away from the second air duct extends into the aromatherapy unit, and a hollow inner cavity of the heating unit acts as a third air duct.

In one embodiment, the aromatherapy storage module further comprises a first energizing unit, and the first energizing unit is connected with the heating unit and used for providing electric power to heat the heating unit.

In one embodiment, the host module comprises a second energizing unit corresponding to the first energizing unit, and when the aromatherapy storage module is installed on the host module, the first energizing unit abuts against the second energizing unit.

In one embodiment, the first housing of the aromatherapy storage module is provided with a first guiding unit, and the support frame of the host module is provided with a second guiding unit matched with the first guiding unit.

In one embodiment, the host module further comprises a power supply unit, and the power supply unit is connected with the second energizing unit.

In one embodiment, the host module comprises a second housing and a third housing, the third housing encloses a groove body to act as the support frame, and when the aromatherapy storage module is installed on the host module, the third housing matches with the first housing.

In one embodiment, the main body of the aromatherapy unit is an oil storage cotton with an air hole, the oil storage cotton fills the storage bin, and the wind generated by the fan unit enters the atomization bin through the first air duct, the second air duct, the third air duct and the oil storage cotton.

In one embodiment, the first guiding unit is a fin higher than a surface of the first housing.

In one embodiment, the second guiding unit is a channel matched with the fin.

In one embodiment, the host module further comprises a circuit board unit.

The aromatherapy device of the present disclosure has the following beneficial effects:
The essential oils atomized by the heating unit or heating wire are diffused into the air by means of wind power. It is easy to carry, and aromatherapy materials or essential oils will not leak. The aromatherapy storage module uses an oil storage cotton to store the aromatherapy materials or essential oils, and the oil storage cotton is contained in the storage bin. A leakage-proof oil-absorbing unit, such as an oil absorbent cotton, is added to the top and bottom of the storage bin to avoid accidental leakage of essential oils during use. The air ducts at the bottom and top of the storage bin are plugged with silicone to prevent evaporation and leakage of aromatherapy materials or essential oils during storage or transportation. In use, this aromatherapy diffuser can be placed at any angle without causing leakage. The storage bin and the atomization bin are combined together, and conventional ultrasonic ceramic atomizers or steel sheet atomizers can easily cause the atomization sheet to be damaged and cannot be used for atomization, and the present disclosure adopts the combination of the storage bin and the atomization bin to become a replaceable aromatherapy storage module, which can avoid the above problem.

Further embodiments of the present disclosure are also capable of achieving other advantageous technical effects that are not listed one by one, which may be partially described below, and which can be expected and understood by those skilled in the art after reading the present disclosure.

### BRIEF DESCRIPTION OF THE DRAWINGS

In order to more clearly illustrate the technical solutions in the embodiments of the present disclosure, the accompanying figures to be used in the description of the embodiments or prior art will be briefly described below. Apparently, the accompanying figures in the following description are only some embodiments of the present disclosure, and to those skilled in the art, other figures may be obtained from these figures without creative labor.

FIG. 1 is a three-dimensional view of the aromatherapy device of the present application;

FIG. 2 is a cross-sectional view of the aromatherapy device of the present application;

FIG. 3 is a three-dimensional view of the host module of the aromatherapy device of the present application;

FIG. 4 is a three-dimensional view of the aromatherapy storage module of the aromatherapy device of the present application; and

FIG. 5 is a cross-sectional view of the aromatherapy storage module of the aromatherapy device of the present application.

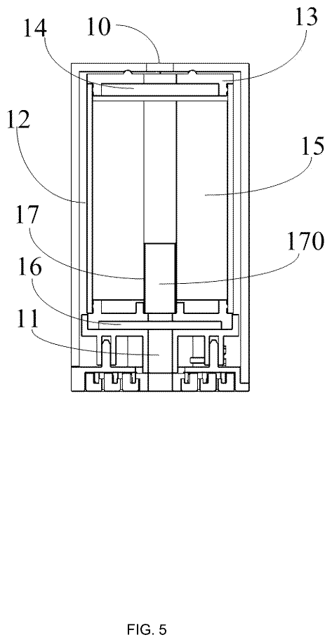

### DETAILED DESCRIPTION

In order to make the purpose, technical solutions and advantages of the embodiments of the present disclosure more clear, the technical solutions in the embodiments of the present disclosure will be clearly and completely described below in conjunction with the figures in the embodiments of the present disclosure. Apparently, the described embodiments are some embodiments of the present disclosure, not all embodiments. The components of the embodiments of the present disclosure, which are usually described and shown in the figures herein, may be arranged and designed in a variety of different configurations.

Therefore, the following detailed description of the embodiments of the present disclosure provided in the figures is not intended to limit the protection scope of the claimed disclosure, but merely indicates selected embodiments of the present disclosure. Based on the embodiments of the present disclosure, all other embodiments obtained by those skilled in the art without making creative labor belong to the protection scope of the present disclosure.

It should be noted that similar signs and letters indicate similar items in the figures below. Thus, once an item has been defined in one of the accompanying figures, it does not need to be further defined and explained in subsequent figures.

In the description of the present disclosure, it should be noted that the orientation or positional relationships indicated by the terms “over”, “under”, “left”, “right”, “vertical”, “horizontal”, “inside”, “outside”, etc. are the orientation or positional relationships based on the accompanying figures, or the orientation or positional relationships in which the products of the present disclosure are customarily placed when in use, and are intended only for the convenience of describing the present disclosure and for simplifying the description, and are not intended to indicate or imply that the devices or elements referred to must be of a particular orientation, be constructed and operated with a particular orientation, and therefore are not to be construed as limitations to the present disclosure. In addition, the terms “first”, “second”, “third”, etc., are used only to distinguish descriptions and should not be construed as indicating or implying relative importance. In the description of the present disclosure, unless otherwise stated, “a plurality of” means two or more.

In the description of the present disclosure, it should also be noted that, unless otherwise expressly specified or defined, the terms “provide” and “connect” should be understood in a broad sense, for example, it may be a fixed connection, or a detachable connection, or an integral connection; it may be a mechanical connection, an electrical connection or a communication connection. For those skilled in the art, the specific meanings of the above terms in the context of the present disclosure may be understood in specific cases.

In the present disclosure, unless otherwise expressly specified or defined, the first feature being “over” or “under” the second feature may include that the first and second features are in direct contact, or comprise that the first and second features are not in direct contact but through another feature therebetween. Besides, the first feature being “over”, “above” and “on” the second feature includes that the first feature is directly above and diagonally above the second feature, or simply indicates that the horizontal height of the first feature is greater than that of the second feature. The first feature being “under”, “below” and “beneath” the second feature includes that the first feature is directly below and diagonally below the second feature, or simply indicates that the horizontal height of the first feature is less than that of the second feature.

As used herein, the terms “install”, “interconnect”, “connect”, “fix”, etc. should be understood in a broad sense, for example, it may be a fixed connection, or a detachable connection, or an integral connection; it may be a mechanical connection, or an electrical connection; it may be a direct connection, or indirection connection through an intermediate medium, or a communication within two elements. For those skilled in the art, the specific meanings of the above terms in the context of the present disclosure may be understood in specific cases.

Embodiments of the present disclosure are described in detail below, and examples of the embodiments are shown in the figures where the same or similar signs from beginning to end indicate the same or similar elements or elements having the same or similar functions. The embodiments described below by reference to the figures are illustrative and are intended to explain the present disclosure only and should not be construed as limitations to the present disclosure.

The present disclosure will be described in more detail below with reference to specific embodiments of the present disclosure.

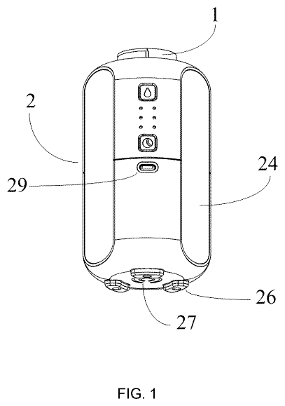

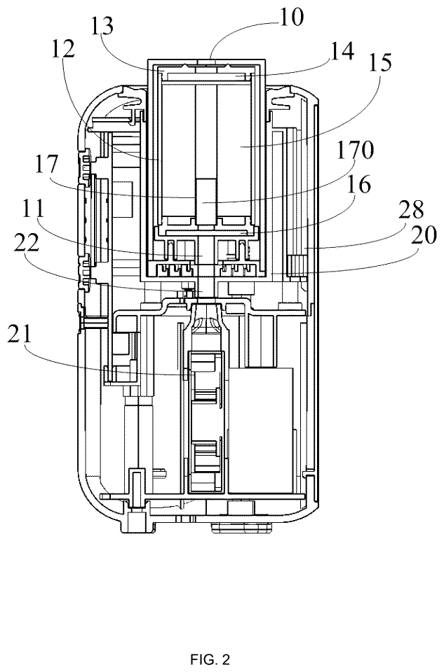

As shown in FIG. 1, the aromatherapy device comprises detachably connected two parts, the first part is an aromatherapy storage module 1, and the second part is a host module 2. The aromatherapy storage module 1 can be primarily configured with an aromatherapy material and can be replaced and maintained as an independent unit. When the aromatherapy material in the aromatherapy storage module 1 is used up, the user can continue the use by replacing the aromatherapy storage module 1 without having to replace the higher-value host module 2. As shown in FIG. 2, the aromatherapy storage module 1 comprises a first opening 10 for diffusing aromatherapy vapor, and the first opening 10 is provided on the top of the aromatherapy storage module 1 and is available for the heat-atomized aromatherapy material to be diffused into the air. Of course, the first opening 10 may also be provided at other positions of the aromatherapy storage module 1. The host module 2 comprises a support frame 20 and a fan unit 21 that are matched with the aromatherapy storage module 1. The support frame 20 is used for supporting or matching the aromatherapy storage module 1 to install the aromatherapy storage module 1 on the host module 2. As shown in FIG. 2, the support frame 20 may be a recess or accommodation space formed by recessing inward relative to the housing of the host module 2. The fan unit 21 is provided inside the host module 2 and is connected with the power supply to send wind into the aromatherapy storage module 1 to diffuse aromatherapy vapor. Wherein, the aromatherapy storage module 1 is installed to the host module 2 to form an air duct unit and an energizing unit, and the air duct unit is configured for the passage of wind generated by the fan unit 21.

As shown in FIG. 2, the host module 2 comprises a first air duct 22, and the wind generated by the fan unit 21 enters the aromatherapy storage module 1 through the first air duct 22. The aromatherapy storage module 1 further comprises a second air duct 11 opposite the first air duct 22. The first air duct 22 and the second air duct 11 constitute a part or all of the air duct unit, so that the wind generated by the fan unit 21 can enter the aromatherapy storage module 1 through the air duct unit (the first air duct 22 and the second air duct 11).

As shown in FIG. 2, the aromatherapy storage module 1 further comprises a storage bin 12 and an atomization bin 13. The storage bin 12 is used for storing an aromatherapy material. The atomization bin 13 is provided above the storage bin 12 and is available for the passage of atomized vapor. After the aromatherapy material in the storage bin 12 is heated and produces vapor, the vapor enters the atomization bin 13 under the effect of the fan unit 21, and is diffused into the air through the first opening 10 on the atomization bin 13.

As shown in FIG. 2, the aromatherapy storage module 1 further comprises a first oil-absorbing unit 14 provided at one end of the storage bin 12, an aromatherapy unit 15 provided in the storage bin 12, a second oil-absorbing unit 16 provided at the other end of the storage bin 12 and opposite to the first oil-absorbing unit 14, a heating unit 17 for heating the aromatherapy unit 15 and extending into the storage bin 12 and a first housing 18, the first oil-absorbing unit 14, the aromatherapy unit 15, the second oil-absorbing unit 16 and the heating unit 17 are provided inside the first housing 18. The aromatherapy unit 15 is a porous material that stores an aromatherapy material or essential oil. The first air duct 22 and the second air duct 11 constitute a part or all of the air duct unit, so that the wind generated by the fan unit 21 can enter the aromatherapy unit 15 in the aromatherapy storage module 1 through the air duct unit (the first air duct 22 and the second air duct 11). The aromatherapy material or essential oil in the aromatherapy unit 15 evaporates under the heating of the heating unit 17 and is brought into the atomization bin 13 under the action of wind.

The provision of the first oil-absorbing unit 14 and the second oil-absorbing unit 16 can avoid the leakage of the aromatherapy materials or essential oils. The aromatherapy storage module 1 uses an oil storage cotton to store the aromatherapy materials or essential oils, and the oil storage cotton is contained in the storage bin 12. A leakage-proof oil-absorbing unit, such as an oil absorbent cotton, is added to the top and bottom of the storage bin 12 to avoid accidental leakage of essential oils during use. The air ducts at the bottom and top of the storage bin are plugged with silicone to prevent evaporation and leakage of the aromatherapy materials or essential oils during storage or transportation. In use, this aromatherapy diffuser can be placed at any angle without causing leakage.

As shown in FIG. 2, the heating unit 17 is a hollow round pipe, for example, a copper tube. One end of the heating unit 17 is communicated with the second air duct 11, one end of the heating unit 17 away from the second air duct 11 extends into the aromatherapy unit 15, and a hollow inner cavity of the heating unit 17 acts as a third air duct 170. The first air duct 22, the second air duct 11 and the third air duct 170 constitute a part or all of the air duct unit, so that the wind generated by the fan unit 21 can enter the aromatherapy storage module 1 through the air duct unit (the first air duct 22, the second air duct 11 and the third air duct 170). The main body of the aromatherapy unit 15 is an oil storage cotton with an air hole, the oil storage cotton fills the storage bin 12, and the wind generated by the fan unit 21 enters the atomization bin 13 through the first air duct 22, the second air duct 11, the third air duct 170 and the oil storage cotton.

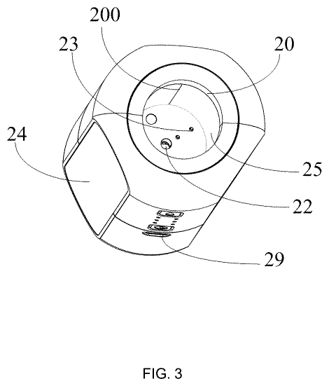

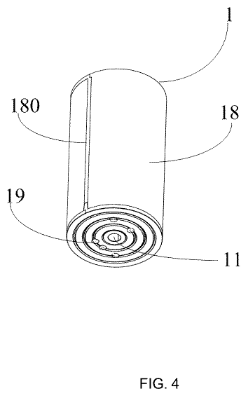

As shown in FIG. 4, the aromatherapy storage module 1 further comprises a first energizing unit 19, and the first energizing unit 19 is connected with the heating unit 17 and used for providing electric power to heat the heating unit 17. As shown in FIG. 3, the host module 2 comprises a second energizing unit 23 corresponding to the first energizing unit 19. When the aromatherapy storage module 1 is installed on the host module 2, the first energizing unit 19 abuts against the second energizing unit 23. The first energizing unit 19 and the second energizing unit 23 constitute an energizing unit. The energizing unit is configured to communicate the power supply line of the host module 2 with the energized line of the aromatherapy storage module 1 through a power supply, and provides electric power for the heating of the heating unit 17. Therefore, the host module 2 further comprises a power supply unit 29, the power supply unit 29 is connected with the second energizing unit 23. As shown in FIG. 2, the host module 2 further comprises a circuit board unit 28. The circuit board unit 28 is used for controlling the fan unit 21, the heating unit 17, etc.

As shown in FIG. 4, the first housing 18 of the aromatherapy storage module 1 is provided with a first guiding unit 180. As shown in FIG. 3, the support frame 20 of the host module 2 is provided with a second guiding unit 200 matched with the first guiding unit 180. The first guiding unit 180 is a fin higher than a surface of the first housing 18. The second guiding unit 200 is a channel matched with the fin. When the user installs the aromatherapy storage module 1 to the host module 2, the first guiding unit 180 is aligned with the second guiding unit 200 and causes the aromatherapy storage module 1 to slide along the second guiding unit 200 into the support frame 20 until the aromatherapy storage module 1 is installed in place.

As shown in FIG. 3, the host module 2 comprises a first housing 24 and a third housing 25, the third housing 25 encloses a groove body to act as the support frame 20, and when the aromatherapy storage module 1 is installed on the host module 2, the third housing 25 matches with the first housing. As shown in FIG. 1, a plurality of legs 26 are also provided at the bottom of the host module 2, and an air inlet 27 is provided on the host module 2 for outside air to enter the inside of the host module 2.

The above are only preferred embodiments of the present disclosure and are not intended to limit the present disclosure, and any modifications, equivalent substitutions and improvements, etc., made within the spirit and principles of the present disclosure shall be included in the protection scope of the present disclosure.

1. An aromatherapy device, wherein the aromatherapy device comprises detachably connected two parts, the first part being an aromatherapy storage module, the aromatherapy storage module comprising a first opening for diffusing aromatherapy vapor;
the second part being a host module, the host module comprising a support frame and a fan unit that are matched with the aromatherapy storage module;
wherein, the aromatherapy storage module is installed on the host module to form an air duct unit and an energizing unit, the air duct unit being configured for the passage of wind generated by the fan unit.

2. The aromatherapy device according to claim 1, wherein the host module comprises a first air duct, through which first air duct the wind generated by the fan unit enters the aromatherapy storage module.

3. The aromatherapy device according to claim 2, wherein the aromatherapy storage module further comprises a storage bin and an atomization bin.

4. The aromatherapy device according to claim 3, wherein the aromatherapy storage module further comprises a first oil-absorbing unit provided at one end of the storage bin, an aromatherapy unit provided in the storage bin, a second oil-absorbing unit provided at the other end of the storage bin and opposite to the first oil-absorbing unit, a heating unit for heating the aromatherapy unit and extending into the storage bin and a first housing, the first oil-absorbing unit, the aromatherapy unit, the second oil-absorbing unit and the heating unit being provided inside the first housing.

5. The aromatherapy device according to claim 4, wherein the aromatherapy storage module further comprises a second air duct opposite to the first air duct.

6. The aromatherapy device according to claim 5, wherein the heating unit is a hollow round pipe, one end of the heating unit is communicated with the second air duct, one end of the heating unit away from the second air duct extends into the aromatherapy unit, a hollow inner cavity of the heating unit acts as a third air duct, a main body of the aromatherapy unit is an oil storage cotton with an air hole, the oil storage cotton fills the storage bin, and the wind generated by the fan unit enters the atomization bin through the first air duct, the second air duct, the third air duct and the oil storage cotton.

7. The aromatherapy device according to claim 6, wherein the aromatherapy storage module further comprises a first energizing unit, the first energizing unit being connected with the heating unit and used for providing electric power to heat the heating unit.

8. The aromatherapy device according to claim 7, wherein the host module comprises a second energizing unit corresponding to the first energizing unit, when the aromatherapy storage module is installed on the host module, the first energizing unit abuts against the second energizing unit, and the host module further comprises a power supply unit, the power supply unit being connected with the second energizing unit.

9. The aromatherapy device according to claim 8, wherein the first housing of the aromatherapy storage module is provided with a first guiding unit, the support frame of the host module is provided with a second guiding unit matched with the first guiding unit, the first guiding unit is a fin higher than a surface of the first housing, and the second guiding unit is a channel matched with the fin.

10. The aromatherapy device according to claim 9, wherein the host module comprises a second housing and a third housing, the third housing encloses a groove body to act as the support frame, and when the aromatherapy storage module is installed on the host module, the third housing matches with the first housing.
